SEQUENCE LISTING (1 sequence)

SEQ ID NO: 1 - DNA, 11773 bp, artificial
  pPB-T11-GFP-IRES-TK-EF1a-rtTA-IRES-puro
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggggcgcgccattctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagcatgggcccatctcgagtctagaagatgggcgggagtcttctgggcaggcttaaaggctaacctggtgtgtgggcgttgtcctgcaggggaattgaacaggtgattaccctgttatccctatgggaagacaatagcaggcaataacttcgtatagcatacattatacgaagttatgcggccggggatcctctagagtcgacgccgcggccgatccgacatcgttaacgcggccgcgctagcttaccggtaactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagtcgagaatcgaattatccatggaatctctggctaactagagaacccactgcttactggcttatcgaaattaatacgactcactatagggagacccaagctggctagttaagctatcaccaactttgtacaaaaaagcaggctatgaggaactcgtttagtgaaccgtcagatcgcctggagacgccatccacgctgttttgacctccatagaagacaccgactctactagaggatctgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataatgtacaagtagcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaactagacccagctttcttgtacaaagttggtgatctagagggcccgcggttcgaaggtaagcctatccctaaccctctcctcggtctcgattctacgcgctcaggtacaagtaaagcggccgctaagatccagacatgataagatacattgatgagtttggacaaaccaaaactagaatgcagtgaaaaaaatgccttatttgtgaaatttgtgatgctattgccttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagatccttcgccggcgattacctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaagttcctattctctagaaagtataggaacttctcgagtctagaagatgggcgggagtcttctgggcaggcttaaaggctaacctggtgtgtgggcgttgtcctgcaggggaattgaacaggtgattaccctgttatccctaggcaagacaatagcaggcaataacttcgtatagcatacattatacgaagttatgcggccggggatcctctagagtcgacgccgcggccgatccgacatcaagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcaggtgtcgtgaacacgctaccggtcccggtctcgagaatcgaattatccatggaactctggctaactagagaacccactgcttactggcttatcgaaattaatacgactcactatagggagacccaagctggctagttaagctatcacaagtttgtacaaaaaagcaggccatgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccggccacccagctttcttgtacaaagtggttgatctagagggcccgcggttcgaaggtaagcctatccctaaccctctcctcggtctcgattctacgcgtaccggtcatcatcaccatcaccattgagtttggccgcgactctagatcataatcagccataccacatttgtagaggttttacttgctttaaaaaacctcccacacctccccctgaacctgaaacataaaatgaatgcaattctgcagacggccgcgcgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggccaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggctcgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgctggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagttcgcgtctggaactctagcgcggccgcgttctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaagttcctattctctagaaagtataggaacttctcgagtctagaagatgggcgggagtcttctgggcaggcttaaaggctaacctggtgtgtgggcgttgtcctgcaggggaattgaacaggtgattaccctgttatccctaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcatgtcgacatactagttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatctagtatacgcgtatgcggccgcttaattaatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2017/0298391 A1
(43) Pub. Date: Oct. 19, 2017

(54) CONDITIONAL RESCUE SYSTEM, CELLS, AND METHODS

(71) Applicant: REGENTS OF THE UNIVERSITY OF MINNESOTA, Minneapolis, MN (US)

(72) Inventors: David A. Largaespada, Mounds View, MN (US); Branden S. Moriarity, Shoreview, MN (US); Robert A. Klink, Minneapolis, MN (US)

(21) Appl. No.: 15/491,545

(22) Filed: Apr. 19, 2017

Related U.S. Application Data
(60) Provisional application No. 62/324,758, filed on Apr. 19, 2016.

(51) Int. Cl. C12N 15/90; C12N 15/11; C12N 15/85; C12N 9/12; C12N 9/22; C12N 2310/20

(52) U.S. Cl. CPC C12N 15/907; C12N 9/1211; C12N 9/22; C12N 15/11; C12N 15/85; C12Y 207/01021; C12N 2310/20; C12N 2800/90; C12N 2830/001; C12N 2830/50; C12N 2840/203; C12N 2800/107

(57) ABSTRACT

A conditional rescue system generally includes a gene transfer system, a polynucleotide including a nuclease-resistant target coding region, and a coding region encoding a conditionally-lethal polypeptide. The gene transfer system is effective to integrate into host cell DNA. The polynucleotide including the nuclease-resistant target coding region is under transcriptional control of an inducible promoter. The coding region encoding a conditionally-lethal polypeptide is transcriptionally linked to the target coding region.

### CONTINUING APPLICATION DATA

This application claims the benefit of U.S. Provisional Application Ser. No. 62/324,758, filed Apr. 19, 2016, which is incorporated by reference herein.

### SEQUENCE LISTING

This application contains a Sequence Listing electronically submitted to the United States Patent and Trademark Office via EFS-Web as an ASCII text file entitled “110-05140101_ST25.txt” having a size of 15,754 bytes and created on Apr. 17, 2017. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY

This disclosure describes, in one aspect, a conditional rescue system. Generally, the conditional rescue system includes a gene transfer system, a polynucleotide including a nuclease-resistant target coding region, and a coding region encoding a conditionally-lethal polypeptide. The gene transfer system is effective to integrate into host cell DNA. The polynucleotide including the nuclease-resistant target coding region is under transcriptional control of an inducible promoter. The coding region encoding a conditionally-lethal polypeptide is transcriptionally linked to the target coding region.

In some embodiments, the conditionally-lethal polypeptide can include a drug-inducible polypeptide toxic to the host cell. In some of these embodiments, the drug-inducible toxic polypeptide comprises a viral thymidine kinase, diphtheria toxin, or a drug-inducible caspase-9.

In some embodiments, the nuclease-resistant target coding region corresponds to a coding region endogenous to the host that is targeted for knock out.

In another aspect, this disclosure describes a host cell that includes any embodiment of the conditional rescue system summarized above.

In another aspect, this disclosure describes a method that generally includes introducing any embodiment of the conditional rescue system summarized above into a host cell, treating the cells with a compound that induces the conditionally-lethal polypeptide, thereby killing cells transcribing the nuclease-resistant target coding region and conditionally-lethal polypeptide in the absence of inducer, introducing into the host cell a nuclease system that targets a coding region endogenous to the host that corresponds to the nuclease-resistant target coding region, and inducing expression of the nuclease-resistant target coding region while the nuclease system inactivates the endogenous coding region targeted by the nuclease system.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1(A-G) shows a generalized version of the concept and generation of conditional rescue and conditional-knockout, non-leaky, transposon rescue line (CNTRL) cells to study candidate genes. FIG. 1A. shows exemplary functional validation of a conditional rescue clone by Western blot analysis of FOXR2 wild type and DKO clone with and without addition of doxycycline (adapted from Moriarity et al., 2014, PloS ONE 9:e96114). FIG. 1B. Functional validation of a conditional rescue clone via soft agar colony formation assay in MPNST cells of FOXR2 wild type and DKO clone with and without addition of doxycycline (adapted from Moriarity et al., 2014, PloS ONE 9:e96114). FIG. 1C. Demonstration that IRES-thymidine-kinase-linked EGFP (EGFP-TK) can be used to eliminate ‘leaky’ clones expressing complementary DNA (cDNA) in absence of doxycycline using ganciclovir. The image in the left panel was taken prior to ganciclovir selection; a subset of cells are EGFP positive, even though no doxycycline was included in the media. The three rightmost panels were taken after 10 days, 13 days, and 17 days, respectively, of ganciclovir selection of cells at 10 ng/mL. Ganciclovir is converted to a toxic drug by phosphorylation by the viral thymidine kinase, and EGFP-TK positive cells—i.e. leaky clones—are gradually eliminated from the population. TRE—Tet response element (7 repeats of a tetracycline operator sequence); IRES—internal ribosome entry site; EF1A—Human elongation factor-1 alpha (a constitutive promoter of human origin); rtTA—reverse tet transactivator (binds to a TRE and permits transcription when tetracycline or one of its derivatives (e.g., doxycycline) is present). FIG. 1(D-H) depicts an exemplary method to create CNTRL cell lines. FIG. 1D. Transfect an all-in-one ‘dox-on’ system containing a piggyBac transposon vector and transposase (PB-TS) to generate a stable cell line via puromycin selection.

FIG. 1E. This system is designed to express a gene of interest (GOI) from the Tet operon linked with TK to eliminate ‘leaky’ clones via treatment with ganciclovir. FIG. 1F. Stable, non-leaky cells are then treated with CRISPR to knockout the endogenous GOI, while doxycycline is added to supplement the cells with the GOI cDNA. Clones are then isolated and genotyped for knockout of the endogenous GOI. FIG. 1G. CNTRL cells can be used for over expressing the GOI. FIG. 1H. CNTRL cells can additionally or alternatively be used for over expressing complete GOI knockout.

FIG. 2. Plasmid map of pPB-T11-GFP-IRES-TK-EF1a-rtTA-IRES-puro.

### DETAILED DESCRIPTION OF ILLUSTRATIVE EMBODIMENTS

Genome editing allows one to design cells with engineered edits of the genome, which can be used to produce therapeutics that are targeted, robust, and/or devoid of undesirable side effects. The ability to knock out genes—either by modifying a coding region or by modifying a regulatory region that controls expression of a coding region—in a variety of cell types can be used to investigate many aspects of human biology. Success depends, at least in part, on being able to develop cell lines using methods that are scalable and/or robust. Genome editing technologies such as, for example, CRISPR/Cas9, have been improved. However, a persistent problem lies in targeting genes that are essential or that confer a major growth/survival advantage to a cell, since knockout of such a gene will be selected against during the gene targeting procedure.

One approach to this problem involves creating so-called conditional knockouts. The method involves introducing sites—using homologous recombination—for site-specific recombinases around critical parts of a gene. For example, conventional conditional knockouts are typically generated using Cre/LoxP technology and homologous recombination to introduce LoxP sites that flank critical portions of a gene. This technique also requires a course of Cre recombinase expression that can be induced. Therefore, use of so-called “foxed” alleles (for flanking LoxP sites) is time consuming and inefficient, especially for a diverse array of cell types, and especially since both (or all) copies of an endogenous gene must be “foxed.” Thus, the approach has been limited mostly to mouse and human embryonic stem cell and a few other cell lines (Maury et al., 2011, Integr Biol (Camb) 3(7):717-723; Bouabe and Okkenhaug, 2013, Methods Mol Biol 1064-315-336).

Targeting addictive oncogenes or essential genes using a targeted nuclease (e.g., ZFNs, TALENs, or CRISPR/Cas9) to create a knockout cell line can be lethal in some cells. This disclosure describes the development and validation of a conditional rescue system to generate knockout cell lines of target genes harboring an inducible rescue vector. Generally, the conditional rescue system includes a gene transfer system effective to integrate into host cell DNA, a nuclease-resistant target polynucleotide under transcriptional control of an inducible promoter, and a coding region encoding a conditionally-lethal polypeptide transcriptionally linked to the target polynucleotide coding region.

In some embodiments, “transcriptional control” means that expression of a gene or polynucleotide is under the control of a promoter with which it is spatially connected. A promoter may be positioned 5′ (upstream) or 3′ (downstream) of a gene under its control. In some embodiments, “transcriptionally linked” refers to the association of nucleic acid fragments in a single fragment so that the function and/or transcription of one fragment is regulated by or tied to the function and/or transcription of the other fragment. For example, a promoter is transcriptionally linked with a nucleic acid fragment when it is capable of regulating the transcription of that nucleic acid fragment.

Generally, the gene transfer system can include a transposon or a viral integration system. Exemplary transposons include, but are not limited to, piggyBac, Sleeping Beauty, Tn7, TcBuster, Frog Prince, etc. Exemplary transposons also include the transposons enumerated in Table 1 and in Arensburger et al. Genetics. 2011; 188(1):45-57 or a SPACE INVADERS (SPIN) transposon (see, e.g., Pace et al., Proc Natl Acad Sci USA. 2008; 105(44):17023-17028). Alternative, the gene transfer system can be integrated into the genome of a host cell using, for example, a retro-transposon, random plasmid integration, recombinase-mediated integration, homologous recombination mediated integration, or non-homologous end joining mediated integration.

TABLE 1

| | |
|---|---|
| Sequence Name | Accession Number |

GENBANK (sequences available on the World Wide Web at
ncbi.nlm.nih.gov

| | |
|---|---|
| Ac-like | (AAC46515) |
| Ac | (CAA29005) |
| AeBuster1 | (ABF20543) |
| AeBuster2 | (ABF20544) |
| AmBuster1 | (EFB22616) |
| AmBuster2 | (EFB25016) |
| AmBuster3 | (EFB20710) |
| AmBuster4 | (EFB22020) |
| BtBuster1 | (ABF22695) |
| BtBuster2 | (ABF22700) |
| BtBuster3 | (ABF22697) |
| CfBuster1 | (ABF22696) |
| CfBuster2 | (ABF22701) |
| CfBuster3 | (XP_854762) |
| CfBuster4 | (XP_545451) |
| CsBuster | (ABF20548) |
| Daysleeper | (CAB68118) |
| DrBuster1 | (ABF20549) |
| DrBuster2 | (ABF20550) |
| EcBuster1 | (XP_001504971) |
| EcBuster3 | (XP_001503499) |
| EcBuster4 | (XP_001504928) |
| Hermes | (AAC37217) |
| hermit | (LCU22467) |
| Herves | (AAS21248) |
| hobo | (A39652) |
| Homer | (AAD03082) |
| hopper-we | (AAL93203) |
| HsBuster1 | (AAF18454) |
| HsBuster2 | (ABF22698) |
| HsBuster3 | (NP_071373) |
| HsBuster4 | (AAS01734) |
| IpTip100 | (BAA36225) |
| MamBuster2 | (XP_001108973) |
| MamBuster3 | (XP_001084430) |
| MamBuster3 | (XP_001084430) |
| MamBuster4 | (XP_001101327) |
| MmBuster2 | (AAF18453) |
| PtBuster2 | (ABF22699) |
| PtBuster3 | (XP_001142453) |
| PtBuster4 | (XP_527300) |
| Restless | (CAA93759) |
| RnBuster2 | (NP_001102151) |
| SpBuster1 | (ABF20546) |
| SpBuster2 | (ABF20547) |
| SsBuster4 | (XP_001929194) |
| Tam3 | (CAA38906) |
| TcBuster | (ABF20545) |
| Tol2 | (BAA87039) |
| tramp | (CAA76545) |
| XtBuster | (ABF20551) |

ENSEMBL (sequences available on the World Wide Web at
ensembl.org)

| | |
|---|---|
| PtBuster1 | (ENSPTRG00000003364) |

REPBASE (sequences available on the World Wide Web at
girinst.org)

| | |
|---|---|
| Ac-like2 | (hAT-7_DR) |
| Ac-like1 | (hAT-6_DR) |
| hAT-5_DR | (hAT-5_DR) |
| MlBuster1 | (hAT-4_ML) |
| Myotis-hAT1 | (Myotis-hAT1) |
| SPIN_Et | (SPIN_Et) |
| SPIN_Ml | (SPIN_Ml) |
| SPIN-Og | (SPIN-Og) |

TEFam (sequences available on the World Wide Web at
tefam.biochem.vt.edu)

| | |
|---|---|
| AeHermes1 | (TF0013337) |
| AeBuster3 | (TF001186) |
| AeBuster4 | (TF001187) |
| AeBuster5 | (TF001188) |
| AeBuster7 | (TF001336) |
| AeHermes2 | (TF0013338) |
| AeTip100-2 | (TF000910) |
| Cx-Kink2 | (TF001637) |
| Cx-Kink3 | (TF001638) |
| Cx-Kink4 | (TF001639) |
| Cx-Kink5 | (TF001640) |
| Cx-Kink7 | (TF001636) |
| Cx-Kink8 | (TF001635) |

The nuclease-resistant target polynucleotide may be placed under the inducible transcriptional control of any suitable inducible system or combination of inducible systems. Exemplary inducible systems include, but are not limited to, any bacterial operon effective in mammalian cells or a synthetic system that involves a DNA binding domain (e.g., CAS9, ZFN, TALEN) fused to a trans-activating domain. In some embodiments, the target polynucleotide may be made nuclease resistant by introduction of silent mutations at a nuclease target site in the target polynucleotide. For example, when the nuclease is a TALEN, the target polynucleotide may be made nuclease resistant by introduction of silent mutations in a TALEN target site.

In some embodiments, a bacterial operon can include a Tet operon or Tet response element (TRE) including, for example, a reverse tet transactivator (rtTA) inducible with tetracycline or one of its derivatives (e.g., doxycycline). In some embodiments, a bacterial operon can be inducible with cumate.

The conditionally-lethal polypeptide can be any polypeptide that, when expressed by a cell, is lethal to the cell. Exemplary conditionally-lethal polypeptides include a viral thymidine kinase (e.g., from herpes simplex virus), diphtheria toxin, a drug-inducible caspase-9, or any other drug-inducible lethal polypeptide that functions in mammalian cells.

The conditional rescue system also can include a selectable marker. In some embodiments, the selectable marker can confer resistance to an antibiotic such as, for example, ampicillin, chloramphenicol, kanamycin, tetracyclin, puromycin, neomycin, a phleomicin, blasticidin, or hygromycin. In other embodiments, the selectable marker can involve, for example, a hyperactive dihydrofolate reductase (DHFR).

The conditional rescue system also can include a visual (including a visualizable) marker including for example, a fluorescent protein (e.g., GFP, EGFP, BFP, CFP, YFP, etc.). In some embodiments, the visual marker may be used to identify a leaky cell by determining if a cell or subset of cells is expressing the visual marker in the absence of induction.

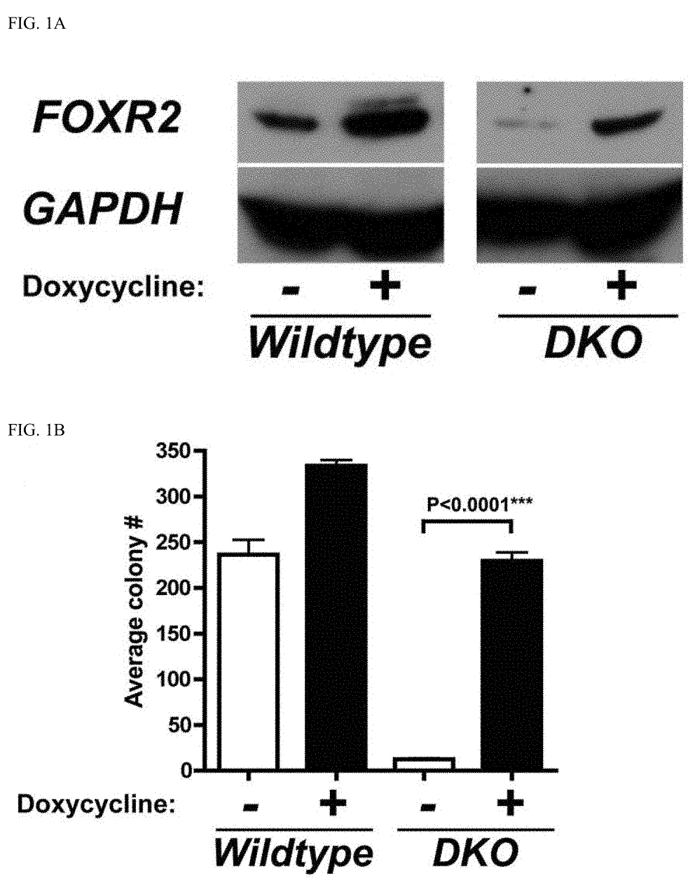

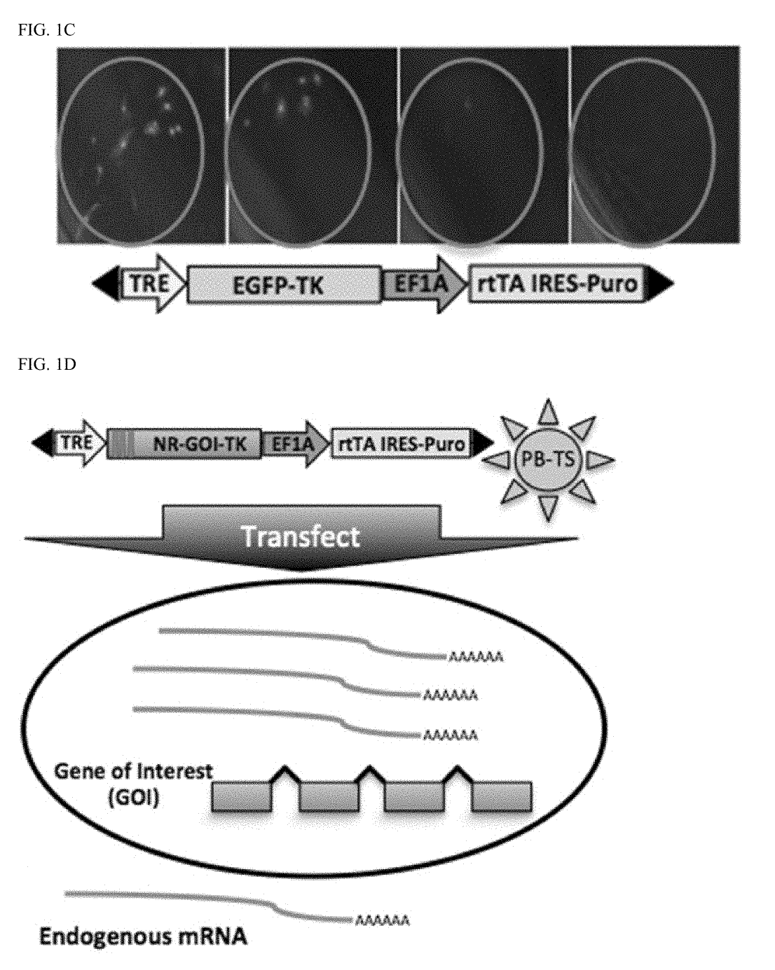

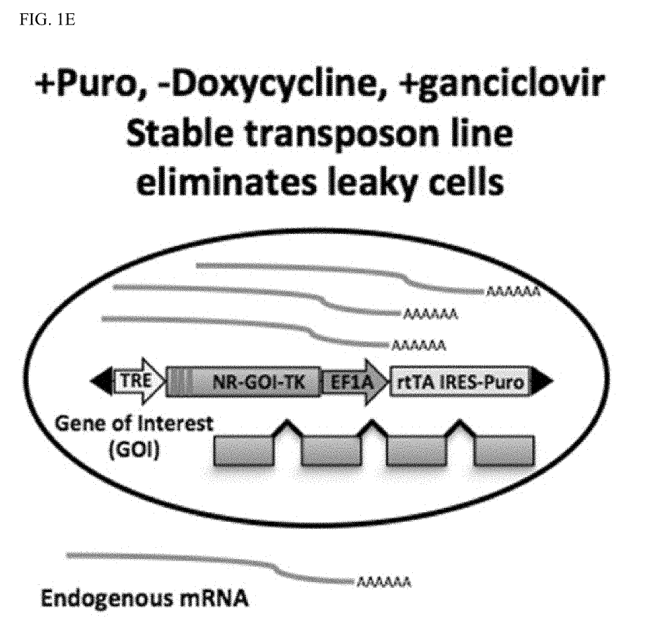

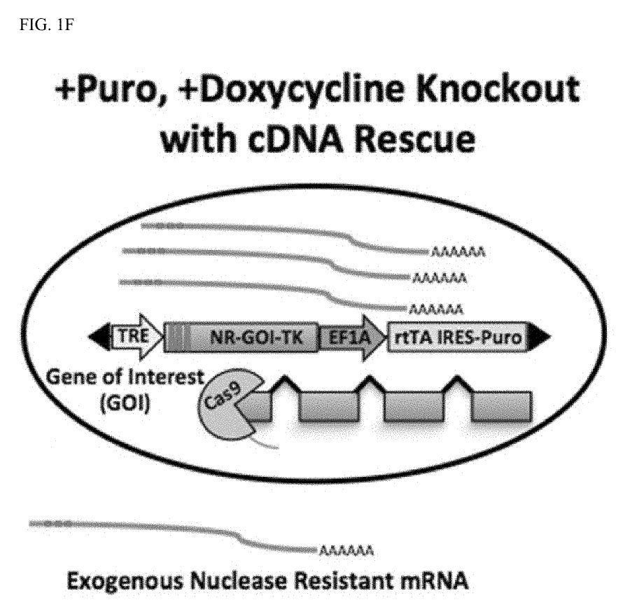

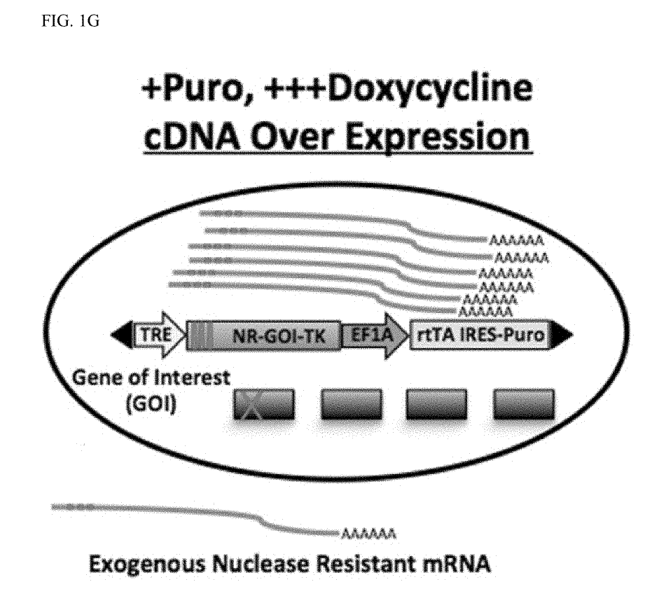

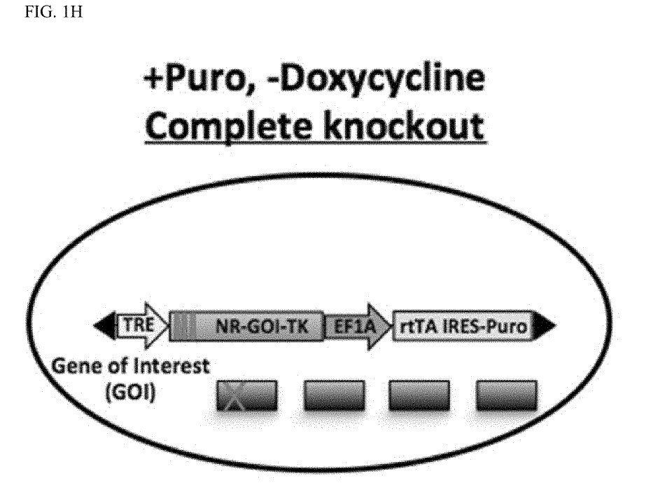

In the exemplary embodiment illustrated in FIG. 1, the approach uses an “all-in-one” doxycycline inducible piggyBac transposon vector to express a nuclease resistant cDNA (NR-cDNA) of the target polynucleotide coding region. The system further contains a selectable marker, puromycin resistance. A cDNA can be made nuclease resistant by introducing a silent mutation at the nuclease target site in the cDNA. Alternatively, the endogenous gene can be targeted with two cut sites that flank the gene (or critical portions of the gene), resulting in the isolation of deletion clones. In this case, the target sites for cutting can be chosen so that they do not cut in cDNA sequences present in the cDNA rescue vector.

The functionality of this rescue approach was demonstrated by targeting the oncogene FOXR2 in malignant peripheral nerve sheath tumors (MPNST) cell lines (FIG. 1A and FIG. 1B). An all-in-one doxycycline-inducible piggyBac transposon was used to express a TALEN resistant cDNA (TR-cDNA) in addition to a puromycin resistance gene. FOXR2 deficient (DKO) clones that dependably induced TALEN resistant-FOXR2 cDNA expression upon treatment with doxycycline were identified by Western blot analysis (FIG. 1A). Loss of FOXR2 in MPNST cells substantially reduces the cells' ability to form colonies in soft agar. Upon treatment with doxycycline, cells were able to form colonies, indicating FOXR2 expression, but colonies were nearly undetectable in the absence of TR-FOXR2 induction (FIG. 1B).

Another problem with using an inducible system in cell lines is that all the existing reagents have some degree of ‘leakiness,’ or non-induced expression of the target polynucleotide in the absence of the inducer. Other systems for conditional gene expression present similar problems. The problem of ‘leakiness’ is mitigated in the conditional rescue cell lines described herein. The cell lines can exhibit minimal—in some cases, no—expression of the rescue polynucleotide in the absence of an inducer, thus allowing one complete control over transgene expression.

This disclosure describes, in another aspect, a method for producing cells in which tightly controlled expression of a rescue copy of an edited/knocked out gene is provided. The method described herein can be used to isolate cells with genes knocked out. Providing the tightly-controlled rescue copy of the knocked out gene overcomes the problem of knockouts that cause cell lethality and/or reduced fitness. Unlike previous methods, the method described herein allows one to reliably produce cells in which expression of the rescue cDNA is expressed if, and only if, the inducer is added to the cells. In this way, the true phenotype of the cells can be ascertained in the absence of gene expression.

The method involves a coding sequence encoding a conditionally toxic polypeptide immediately following the nuclease-resistant target polynucleotide (e.g., a cDNA). The nuclease-resistant target polynucleotide corresponds to the host cell target polynucleotide that is being knocked out. Thus, during clone isolation, a step can be included after gene transfer in which all clones that express the nuclease-resistant target polynucleotide in the absence of the inducer are eliminated by activating the conditionally lethal gene product. In one embodiment, the conditionally lethal gene product is the Herpes Simplex Type I Thymidine Kinase (HSV-TK) gene. The addition of ganciclovir kills ‘leaky’ clones—i.e., cells that express the nuclease-resistant target polynucleotide and HSV-TK in the absence of inducer. As an alternative, there are other proteins that cause lethality only when some condition—e.g., a chemical inducer—is met. In the embodiment illustrated in FIG. 1, an IRES-thymidine kinase (TK) element is included following the cDNA. This allows for robust killing of ‘leaky’ cDNA expressing clones prior to nuclease mediated gene knockout when ganciclovir is present.

FIG. 1C shows a stable population of SJSA-1 human osteosarcoma cells that are resistant to puromycin and harbor the pPB-T11-EGFP-IRES-TK EF1α-rtTA-IRES-Puro transposon vector. Examination of a population of such cells under a fluorescent microscope revealed that a subset of the cells leakily expressed detectable EGFP (FIG. 1C left panel), even though no doxycycline inducer was present in the media. The population of cells was then subjected to gangciclovir selection and examined over time. The three rightmost panels of FIG. 1C, were taken after 10 days, 13 days, and 17 days, respectively, of ganciclovir selection of cells. EGFP positive cells—i.e., leaky clones—are gradually eliminated from the population. These data demonstrate that IRES-thymidine-kinase-linked EGFP (EGFP-TK) can be used to eliminate leaky clones that express cDNA in the absence of doxycycline using prior ganciclovir selection.

FIG. 1D-G illustrates a robust, scalable method to generate genetically engineered cell lines termed as conditional-knockout, non-leaky, transposon rescue line (CNTRL) cells. The process, in one embodiment, begins with generation and transfection of an all-in-one ‘dox-on’ system containing piggyBac transposon vector and transposase (PB-TS) to generate a stable cell line via puromycin selection (FIG. 1D). This system is designed to express a nuclease-resistant target polynucleotide that corresponds to an endogenous target polynucleotide (e.g., gene of interest (GOI)) that is being knocked out. In the illustrated exemplary embodiment, the nuclease-resistant target polynucleotide is expressed from the Tet operon transciptionally linked with TK to eliminate leaky clones through selective treatment with ganciclovir prior to inducing expression of the nuclease-resistant polynucleotide with doxycycline (FIG. 1E). Stable, non-leaky cells are then treated with CRISPR to knockout the endogenous target polynucleotide (e.g., gene of interest), while doxycycline is added to supplement the cells with the nuclease-resistant target polynucleotide (FIG. 1F). Clones are then isolated and genotyped for knock out of the endogenous gene of interest. CNTRL cells can be used for either overexpressing the nuclease-resistant target polynucleotide (FIG. 1G) or complete knockout of the endogenous target polynucleotide (FIG. 1H).

The method and cell constructs described herein have wide-ranging utility for controlling gene expression in general. For example, the cells may be used, as described above, to provide tightly controlled, inducible background expression of a gene knockout in order to maintain viability of the knockout cell line. The cell lines allow one to completely inhibit cDNA expression in the absence of doxycycline and/or control cDNA overexpression by adding excess doxycycline. Thus, one can study knock out and overexpression of a target gene using the same cells.

Moreover, these cells are isogenic. Other methods for gene modulation, such as simple knockout, shRNA, or siRNA knockdown use separate control cell lines (such as non-silencing or scrambled control) that have substantial clone to clone heterogeneity. In contrast, the conditional rescue cells described herein are identical in all respects aside from cDNA induction or suppression via doxycycline, making them an ideal tool for, for example, cancer genetics studies.

Third, the strategy described herein solves the problem of leakiness, a problem that persists even in sophisticated conventional inducible genetic vectors.

Finally, the method described herein is scalable and could be implemented to make libraries of genetically modified cell lines for nearly any human gene, making this technology relevant to, for example, the research and/or pharmaceutical community.

Although described and illustrated herein in the context of an exemplary embodiment in which the emergence of leaky clones is reduced using thymidine kinase expression linked with cDNA expression in a doxycycline-inducible system, the method may be practiced (and cell lines produced) using other selection strategies. TK expression can take 7-10 days to kill leaky cells and may require that the level of leakiness—i.e., TK expression—to be high. Thus, in alternative embodiments, one can use, for example, a drug-inducible caspase-9 (iCaspase-9) selection strategy, as it may require substantially less time for selection (e.g., less than 30 minutes) and less expression of the transgene. iCaspase-9 is an engineered protein modified to be inactive except when induced with the drug AP1903, which activates caspase-9 activity and leads to programmed cell death. Thus, cells can be designed to include an all-in-one doxycycline-inducible transposon vector harboring an IRES-iCaspase-9 element.

While described herein in the context of exemplary embodiments in which the rescue polynucleotide encodes EGFP or FOXR2, the technology described herein can involve a rescue polynucleotide that is capable of rescuing the knockout of any gene of interest. A gene of interest can include, for example, EGFP, FOXR2, CCND1, or G6PD (Moriarity et al., 2014, PLoS ONE 9(5):e961144.). In some embodiments, gene of interest can include an oncogene. In some embodiments, gene of interest can include an essential gene. An essential gene can include, for example, a gene identified by the Database of Essential Genes, available on the world wide web at tubic.tju.edu.cn/deg/.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLE

### Design and Construction of a Conditionally Expressed EGFP-IRES-TK Transposon Vector.

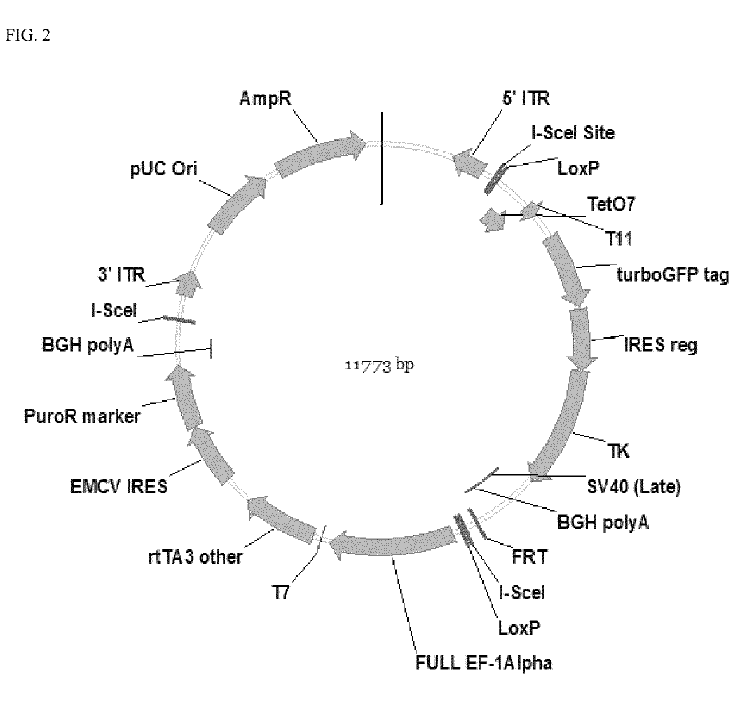

A map of pPB-T11-EGFP-IRES-TK-EF1a-rtTA-IRES-Puro vector (SEQ ID NO:1) is shown in FIG. 2. This plasmid contains a piggyBac transposon vector into which the following sequence elements were cloned: a T11 tet-response element (TRE) (T11), an enhanced green fluorescent protein (EGFP) coding sequence, an internal ribosome entry site (IRES), the Herpes Simplex Virus Thymidine Kinase gene (TK) followed by a polyadenylation (polyA) site, the human EF1a promoter, the reverse tet transactivator (rtTA), an IRES, puromycin resistance gene (Puro), and a final polyadenylation site. All these elements are flanked by inverted terminal repeats (ITRs) for the piggyBac transposase.

### Cell Culture

SJSA-1 cells (ATCC, Manassas, Va.) were cultured in RPMI (ATCC, Manassas, Va.) media with 10% FBS at 37° C. with 5% CO₂. Transfection was conducted by electroporation using the Neon Electroporation System (Invitrogen, ThermoFisher Scientific, Inc., Waltham, Mass.). 500 ng of the plasmid was used to transfect 200,000 SJSA-1 cells. Cells were selected in 1 μg/ml of puromycin (Gibco, ThermoFisher Scientific, Inc., Waltham, Mass.). Stable populations of puromycin-resistant cells were visualized for EGFP expression before and during selection in media including 10 μg/mL ganciclovir (InvivoGen, San Diego, Calif.) to eliminate leaky clones.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A conditional rescue system comprising:
a gene transfer system effective to integrate into host cell DNA;
a polynucleotide comprising a nuclease-resistant target coding region under transcriptional control of an inducible promoter; and
a coding region encoding a conditionally-lethal polypeptide transcriptionally linked to the target coding region.

2. The conditional rescue system of claim 1 wherein the gene transfer system comprises a transposon or a viral integration system.

3. The conditional rescue system of claim 2 wherein the transposon comprises piggyBac, Sleeping Beauty, Tn7, TcBuster, Frog Prince, a SPIN transposon, or a transposon identified in Table 1.

4. The conditional rescue system of claim 1 wherein the inducible promoter is inducible with doxycycline or cumate.

5. The conditional rescue system of claim 1 wherein the inducible promoter comprises a synthetic fusion comprising a DNA binding domain and a trans-activation domain.

6. The conditional rescue system of claim 5 wherein the DNA binding domain comprises Cas9, ZFN, or TALEN.

7. The conditional rescue system of claim 1 wherein the conditionally-lethal polypeptide comprises a drug-inducible polypeptide toxic to the host cell.

8. The conditional rescue system of claim 7 wherein the drug-inducible toxic polypeptide comprises a viral thymidine kinase, diphtheria toxin, or a drug-inducible caspase-9.

9. The conditional rescue system of claim 1 further comprising a selectable marker.

10. The conditional rescue system of claim 9 wherein the selectable marker comprises resistance to an antibiotic.

11. The conditional rescue system of claim 10 wherein the antibiotic comprises puromycin or neomycin.

12. The conditional rescue system of claim 1 wherein the nuclease-resistant target coding region corresponds to a coding region endogenous to the host that is targeted for knock out.

13. A host cell comprising the conditional rescue system of claim 1.

14. The host cell of claim 13 wherein the host cell is or is derived from a mammalian cell.

15. A method comprising:
introducing the conditional rescue system of claim 1 into a host cell;
treating the cells with a compound that induces the conditionally-lethal polypeptide, thereby killing cells transcribing the nuclease-resistant target coding region and conditionally-lethal polypeptide in the absence of inducer;
introducing into the host cell a nuclease system that targets a coding region endogenous to the host that corresponds to the nuclease-resistant target coding region; and
inducing expression of the nuclease-resistant target coding region while the nuclease system inactivates the endogenous coding region targeted by the nuclease system.
